(19) United States
(12) Patent Application Publication
(10) Pub. No.: US 2020/0029982 A1
(43) Pub. Date: Jan. 30, 2020

(54) THREE FILTERS CATHETER FOR BRAIN PROTECTION AND METHOD

(71) Applicant: Luiz Lanat Pedreira de Cerqueira Filho, Salvador (BR)

(72) Inventor: Luiz Lanat Pedreira de Cerqueira Filho, Salvador (BR)

(73) Assignee: Luiz Lanat Pedreira de Cerqueira Filho, Salvador (BR)

(21) Appl. No.: 16/378,244

(22) Filed: Apr. 8, 2019

Related U.S. Application Data
(60) Provisional application No. 62/703,867, filed on Jul. 26, 2018.

(51) Int. Cl. A61B 17/221; A61B 90/00

(52) U.S. Cl. CPC A61B 17/221; A61B 2017/22038; A61B 2017/2215; A61B 90/39

(57) ABSTRACT

A blood filtering catheter comprising three filters for cerebral embolic protection is provided. The catheter further includes at least one guidewire lumen. The catheter further includes at least one basket-shaped filter. The catheter further includes at least one filter made of a material impermeable to embolic materials. The catheter further includes means to control insertion direction of at least one filter. The catheter further includes at least one fluid lumen in an elongated catheter shaft, a fluid lumen having at least one fluid connection. The catheter further includes a distal end deflectable with respect to longitudinal axis of the catheter in absence of any deflection. The catheter further includes at least one radiological mark. Methods of using a blood filtering catheter for cerebral embolic protection including three filters are provided.

### CROSS-REFERENCE TO RELATED APPLICATIONS

This application claims an invention which was disclosed in a provisional application No. 62/703,867, filed Jul. 26, 2018, entitled “Catheter of three filters and method”. The benefit under 35 USC § 119(e) of the United States provisional application is hereby claimed, and the aforementioned application is hereby incorporated herein by reference.

### DESCRIPTION

### Field of Invention

The invention is in the field of medical devices for cerebral embolic protection and pertains particularly to a blood filtering catheters made to capture and remove clots and debris of the blood going to the brain during intravascular procedures.

### BACKGROUND

In the art of medical devices for cerebral embolic protection many different types of blood filtering catheters have been developed for endovascular use to filter the blood going to the brain. In prior art, a blood filtering catheter for cerebral embolic protection is a blood filtering catheter that filters the blood going to the brain to prevent stroke.

One problem with the traditional blood filtering catheter for cerebral embolic protection is to have only one guidewire lumen which makes difficult the access to more than one cerebral vessel at the same time. Another limitation of the current blood filtering catheters for cerebral embolic protection is to have less than two radiological contrast access which makes difficult to doe arteriography, makes difficult to doe radiological identification of cerebral vessels and makes difficult to correct positioning of the filters in the cerebral arteries.

Current blood filtering catheters for cerebral embolic protection such as with one filter partially protect the patient against stroke. Current blood filtering catheter for cerebral embolic protection with two filters do not filter the blood from the left vertebral artery allowing the passage of debris to the brain.

It has not been reported a catheter to protect three cerebral arteries from clots and debris during or after cardiac or other intravascular treatments.

Therefore, what is clearly needed is a blood filtering catheter comprising three filters for cerebral embolic protection and a method for use that solves the problems mentioned above.

### SUMMARY OF THE INVENTION

In one embodiment of the invention a blood filtering catheter comprising three filters for cerebral embolic protection is provided. Also, in other embodiment the invention further comprising at least one guidewire lumen. Also, in other embodiment the invention further comprising at least one basket shaped filter. Also, in other embodiment of the invention further comprising at least one filter made of a material impermeable to embolic materials. Also, in other embodiment the invention further comprising means to control the insertion direction of at least one filter. Also, in other embodiment the invention further comprising at least one fluid lumen in the elongated catheter shaft, the fluid lumen having at least one fluid connection. Also, in other embodiment the invention further comprising at least one distal end of the catheter deflectable with respect to longitudinal an axis of the catheter in the absence of any deflection. Also, in other embodiment the invention further comprising at least one radiological mark.

In other aspect of the invention a method for cerebral embolic protection using a three filters catheter, comprising: (a) deploying a first filter in a first cerebral artery, (b) deploying a second filter in a second cerebral artery, and (c) deploying a third filter in a third cerebral artery, is provided.

Also, in other aspect of the invention, a method for cerebral embolic protection using a three filters catheter, comprising: (a) removing a first filter of a first cerebral artery, (b) removing a second filter of a second cerebral artery, and (c) removing a third filter of a third cerebral artery, is provided.

### BRIEF DESCRIPTION OF THE DRAWINGS

FIG. 1 is a side view of an embodiment of the invention.

FIG. 2 is a side view of a punch set 35, according to an embodiment of the invention.

FIG. 3 is a side view of a three filters catheter 34 ready to be inserted into the sleeve 32, according to an embodiment of the invention.

FIG. 4 is a side view of an embodiment of a distal end of the invention.

FIG. 5 is a side view of the distal end of a three filters catheter 34 for cerebral embolic protection, according to an embodiment of the invention.

FIG. 6 is a cross-section view of an aortic conduit 20, according to an embodiment of the invention.

FIG. 7 is a side view the first filter 3, according to an embodiment of the invention.

FIG. 8 is a perspective view of a segment of the three filters catheter 34 with the third filter 1 in open position, according to an embodiment of the invention.

FIG. 9 is a side cross-sectional view of the second filter 2 positioned within the left carotid artery 14, according to an embodiment of the invention.

FIG. 10 is an illustration of the invention being used in the living body according to an embodiment of the invention.

FIG. 11 is an illustration of other embodiment of the invention showing the first filter 3 deployed in the left subclavian artery 17.

FIG. 12 is an illustration of other embodiment of the invention in which the first guidewire 10 is positioned in the left carotid artery 14 with the third filter 1 deployed in the left subclavian artery 17.

FIG. 13 is an illustration of other embodiment of the invention showing the second filter 2 deployed in the left carotid artery 14, and with the third filter 1 deployed in the left subclavian artery 17.

FIG. 14 is an illustration of other embodiment of the invention showing the first filter 3 deployed in the brachiocephalic trunk 16, the second filter 2 deployed in the left carotid artery 14, and with the third filter 1 deployed in the left subclavian artery 17.

FIG. 15 is a side view of other embodiment of the invention.

### DETAILED DESCRIPTION OF THE PREFERRED EMBODIMENTS

The inventor provides a blood filtering catheter comprising three filters for cerebral embolic protection that protects against the risk of stroke. The new blood filtering catheter comprising three filters for cerebral embolic protection captures and removes blood clots and fragments that go to the brain in three different cerebral arteries during intravascular procedures, one filter is deployed in one cerebral artery, the second filter is deployed in a second cerebral artery, and a third filter is deployed in a third cerebral artery. A single blood filtering catheter comprising three filters for cerebral embolic protection allows filtration of all cerebral blood flow. The invention is described in enabling detail in the following examples, which may represent more than one embodiment of the invention, together with the accompanying drawings in which like numerals represent similar components.

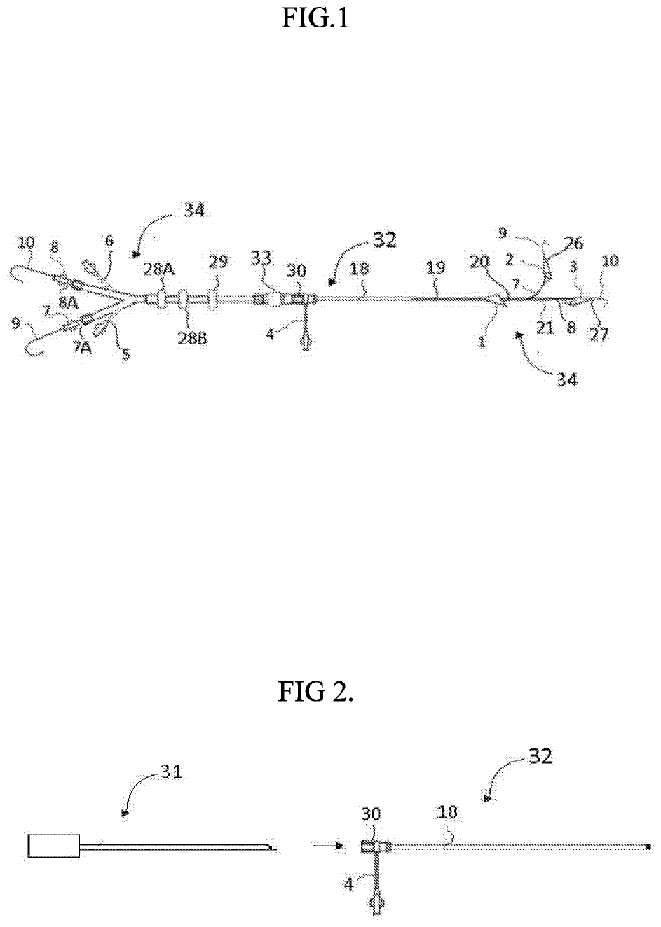

The preferred embodiments according to the invention are shown in FIG. 1-15.

FIG. 1 is a side view of an embodiment of the invention. A three filters catheter 34 is engaged in a sleeve 32 forming a single set. A third filter 1 is made of a material impermeable to embolic materials, a second filter 2 is made of a material impermeable to embolic materials, a first filter 3 is made of a material impermeable to embolic materials. In this example the third filter 1 is open, the second filter 2 is open, and the first filter 3 is open. A third handle 29 longitudinal sliding promotes the opening and closing of the third filter 1. A second handle 28B longitudinal sliding promotes the opening and closing of the second filter 2. A first handle 28A longitudinal sliding promotes the opening and closing of the first filter 3. The third filter 1 is made to be stored closed within a second sheath 19. The second filter 2 and the first filter 3 are made to be stored closed within a third sheath 21. The three filters catheter 34 includes a second guidewire lumen 7A and a first guidewire lumen 8A, for the insertion of two independent guidewires. A first guidewire 10 is inserted into first guidewire lumen 8A. A second guidewire 9 is inserted into second guidewire lumen 7A. A second conduit 7 is deflectable with respect to its longitudinal axis in the absence of any deflection. The second conduit 7 is molded in a curved shape and has a memory effect when rectified to its original curved shape, its insertion direction is controlled by its longitudinal sliding relative to its longitudinal axis and by its longitudinal rotation axis. In one embodiment a first conduit 8 is deflectable with respect to its longitudinal axis in the absence of any deflection, it has a curved shape and has memory effect, when rectified it tends to return to its original curved shape, its insertion direction is controlled by its longitudinal sliding relative to its longitudinal axis and its rotation relative to its longitudinal axis. In one embodiment the third sheath 21 is deflectable with respect to its longitudinal axis in the absence of any deflection, the third sheath 21 is shaped in curved form and has memory effect when rectified tends to return to its original curved shape, and may have its insertion direction controlled by its longitudinal axis sliding relative to its longitudinal axis and its rotation relative to its longitudinal axis, stored within a first sheath 18 is rectified, if slid longitudinally out of the first sheath 18 tends to resume its curved shape. In this example the three filters catheter 34 includes fluids lumen in its elongated shaft having fluid connections: a first radiological contrast access 4, a second radiological contrast access 5, a third radiological contrast access 6. In this example the first radiological contrast access 4 communicates with the first sheath 18 and allows the injection of radiological contrast. The second radiological contrast access 5 communicates with the second guidewire lumen 7A and allows radiological contrast injection in one artery. The third radiological contrast access 6 communicates with the first guidewire lumen 8A to allow radiological contrast injection in one artery. One aspect of the example is the ability to filter the blood in the living body at the same time into three different arteries. One aspect of the example is to allow maintenance of cerebral blood flow throughout a chest or heart procedure, preventing debris from migrating to the brain causing cerebral ischemia and stroke. One aspect of the example is the great advantage of being easy to deploy and easy and quick to remove. One aspect of the example is that it is adapted to be used with one deployed filter by filtering the blood. One aspect of the example is that it is adapted to be used with two deployed filters by filtering the blood. One aspect of the example is that it is adapted to be used with three deployed filters by filtering the blood. In some embodiments each filter is adapted to be deployed and removed independently of the other filters.

One aspect of the example is that the three filters catheter 34 has fluid lumens in the elongated catheter shaft, the first guidewire lumen 8A and the second guidewire lumen 7A are used for guidewire and radiological contrast passage. In this example the fluids lumens have fluid connections for radiological contrast injection, the first radiological contrast access 4, the second radiological contrast access 5, the third radiological contrast access 6. The second radiological contrast access 5 communicates with the second guidewire lumen 7A and allows the injection of radiological contrast. The third radiological contrast access 6 communicates with the first guidewire lumen 8A to allow radiological contrast injection. The first sheath 18, the second sheath 19 and third sheath 21 are independently actuatable. In some example the first handle 28A, the second handle 28B and the third handle 29 are multiple section handle. In some embodiment the first filter 3, the second filter 2 and the third filter 1 are made in different shapes. An embodiment of the three filters are adapted to be in a basket shape, in other embodiment of the three filters, they are adapted to be in a ball shape, in other embodiment of the three filters are adapted to be in a cone shape, in other embodiment of the three filters are adapted to be cylindrical shape but are not limited to them.

In some embodiment the invention comprising at least one radiological mark. In some embodiments the entire catheter is made to be radiologically opaque. In some embodiments any of the catheter parts is made to be radiologically opaque.

FIG. 2 is a side view of a punch set 35, according to an embodiment of the invention. A mandrel 31 is constructed to engage the sleeve 32 in a longitudinally sliding manner forming the single punch set 35. The single punch set 35 is puncturing in one right artery. After the artery puncturing, the mandrel 31 is removing from the sleeve 32 to introduce the three filters catheter 34 into the sleeve 32 lumen. The three filters catheter 34 is progressing into the artery lumen. The first radiological contrast access 4 allows the injection of fluids such as radiological contrast and allows the collection of blood samples.

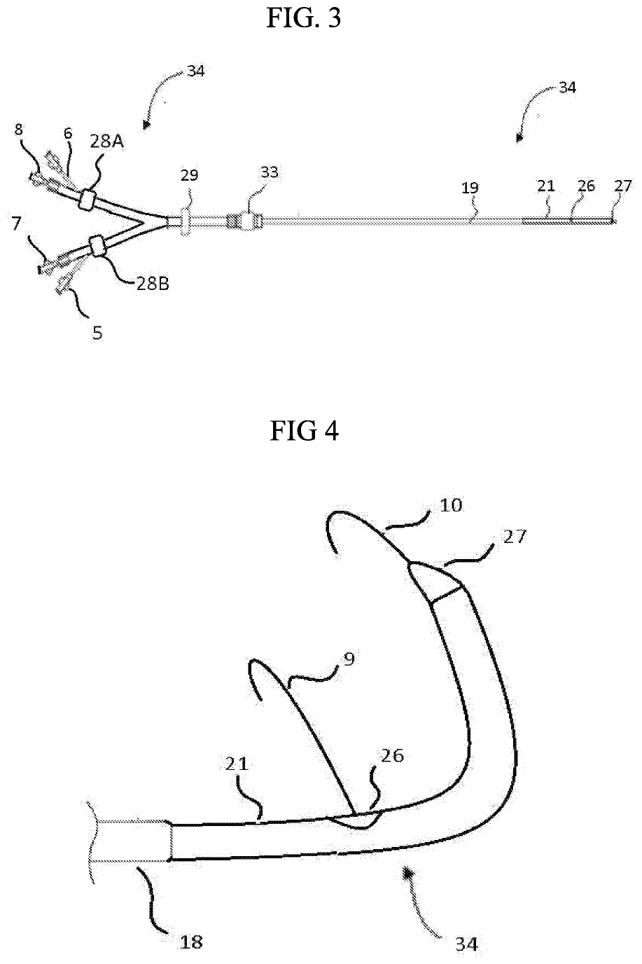

FIG. 3 is a side view of the three filters catheter 34 ready to be inserted into the sleeve 32, according to an embodiment of the invention. The third filter 1 is closing and storing in the second sheath 19, the second filter 2 is closing and storing in the third sheath 21, the first filter 3 is closing in the third sheath 21. Moving longitudinally the third handle 29 drives the opening and closing of the third filter 1, moving longitudinally the second handle 28B drives the opening and closing of the second filter 2, moving longitudinally the first handle 28A drive the opening and closing of the first filter 3. A sealing 33 is made to be moving longitudinally relative to the second sheath 19 to enable it to be engaging to a base 30. The sealing 33 is configured to connecting releasably the base 30 of the sleeve 32.

FIG. 4 is a side view of an embodiment of the distal end of the invention. In this example the distal end of the three filters catheter 34 is deflectable with respect to longitudinal axis of the three filters catheter 34 in the absence of any deflection. The third sheath 21 is deflectable with respect to the longitudinal axis of the three filters catheter 34 in the absence of any deflection, the third sheath 21 is molded in curved shape and has memory effect, when rectified it tends to return to its original curved shape, and its insertion direction may be controlled by its longitudinal sliding relative to its longitudinal axis and by its rotation relative to its longitudinal axis. When the third sheath 21 is stored within the first sheath 18, it is rectified, when it is slid longitudinally out of the first sheath 18, tends to return to its original curved shape. The second filter 2 and the first filter 3 are stored within the third sheath 21. The third sheath 21 has memory effect and its curvature is adapted to be controlled to allow access to an artery.

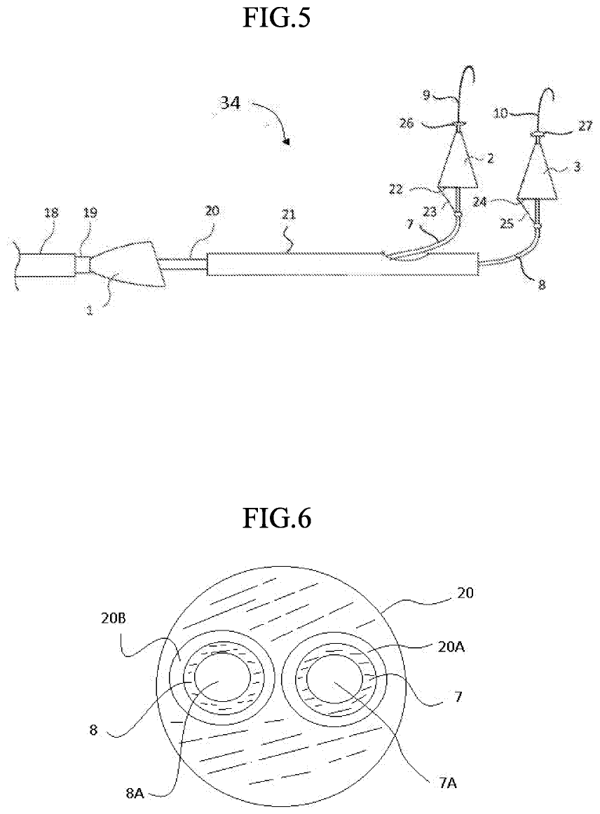

FIG. 5 is a side view of the distal end of a three filters catheter 34 for cerebral embolic protection, according to an embodiment of the invention. The third filter 1 is made of an impermeable material to embolic materials, the second filter 2 is made of an impermeable material to embolic materials, the first filter 3 is made of an impermeable material to embolic materials. The third filter 1 is a basket shaped filter, the second filter e is a basket shaped filter, the first filter 3 is a basket shaped filter. The third filter 1 is open, the second filter 2 is open, the first filter 3 is open. The third filter 1 is made to be stored closed within the second sheath 19. A filter ring 39 assists in the opening of the third filter 1. The second filter 2 and the first filter 3 are made to be stored closed within the third sheath 21. The first guidewire 10 is inserted into the first guidewire lumen 8A. The second guidewire 9 is inserted into the second guidewire lumen 7A.

In this example the distal end of the catheter is deflectable with respect to longitudinal axis of the catheter in the absence of any deflection. The second conduit 7 is molded in a curved shape and has a memory effect when rectified tends to return to its original shape, its insertion direction is controlled by its longitudinal sliding relative to its longitudinal axis and its longitudinal axis rotation. In this example the second conduit 7 is deflectable with respect to its longitudinal axis in the absence of any deflection, is molded in a curved shape and has a memory effect when rectified tends to return to its original shape. The first conduit 8 is deflectable with respect to its longitudinal axis in the absence of any deflection, it has curved shape and has memory effect, when rectified tends to return to its original shape. The third sheath 21 is deflectable with respect to its longitudinal axis in the absence of any deflection, is molded in a curved shape and has a memory effect when rectified tends to return to its original shape.

In this example the three filters catheter 34 comprising means to control the insertion direction of at least one filter. The insertion direction of the first filter 3 is controlled by the longitudinal sliding of the first conduit 8 and the longitudinal sliding of the third sheath 21, relative to its longitudinal axis and by rotation axis. The insertion direction of the second filter 2 is controlled by the longitudinal sliding of the second conduit 7 and the longitudinal sliding of the third sheath 21, relative to longitudinal axis and by rotation axis. In this example the diameter of an aortic conduit 20 is made to allow the third filter 1 to be stored within the second sheath 19. The carotid ring 22 assists to keeps the second filter 2 opened. The support cable 23 keeps the second filter 2 stable and prevents it from rising up with blood pressure. A radiological contrast can be injected from a second guidewire lumen 7A through a tip 26. The brachiocephalic ring 24 assists to keeps the first filter 3 opened. The support cable 25 keeps the first filter 3 stable and prevents it from rising up with blood pressure. A radiological contrast can be injected from first guidewire lumen 8A through a nib 27. According to other embodiment of the present invention.

FIG. 6 is a cross-section view of an aortic conduit 20, according to an embodiment of the invention. Through a first lumen 20A passes the second conduit 7. Through the second guidewire lumen 7A passes the second guidewire 9. Through a second lumen 20B passes the first conduit 8, through the lumen 8A passes the first guidewire 10, according to an embodiment of the invention.

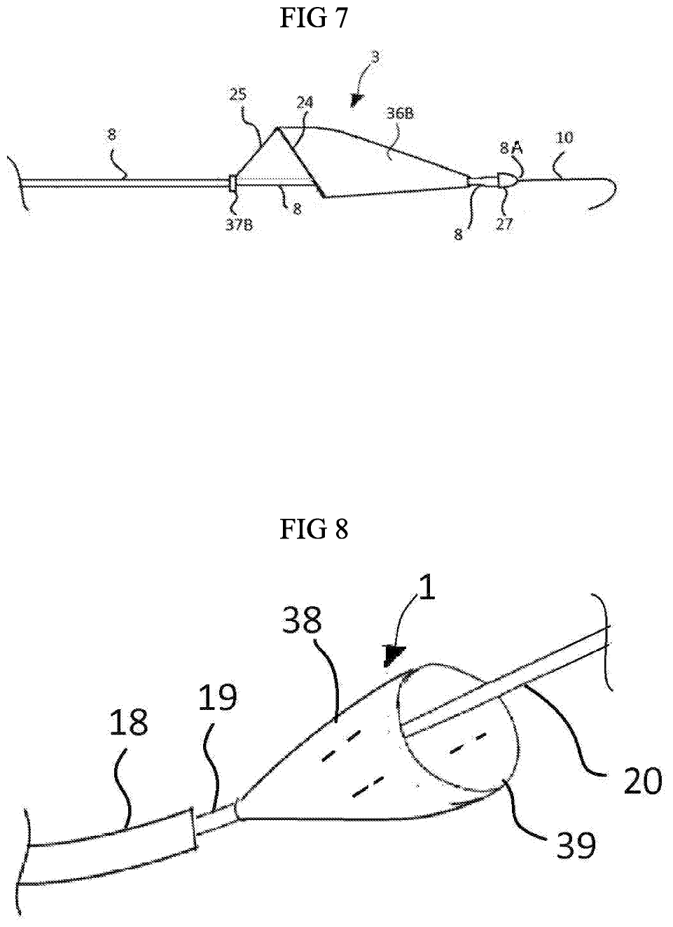

FIG. 7 is a side view of the first filter 3, according to an embodiment of the invention. In this embodiment, the first filter 3 is a basket shaped filter. The brachiocephalic ring 24 assists to keeps the first filter 3 open. The support cable 25 keeps the first filter 3 stable and prevents it from rising up with blood pressure. Radiological contrast can be injected from the first guidewire lumen 8A through the nib 27. A holder fixture 37B secures the support cable 25 in the first conduit 8. A second mesh 36B is permeable to blood and is impermeable to embolic materials. The first guidewire 10 is inserted into the first guidewire lumen 8A.

FIG. 8 is a perspective view of a segment of the three filters catheter 34 with the third filter 1 in open position. The filter ring 39 helps to keep the third filter 1 open. A third mesh 38 is made of a material permeable to blood and impermeable to embolic materials. After filtering the blood, the third filter 1 contains embolic materials. In one embodiment, the third filter 1 is closed and stored within the first sheath 18 by longitudinal sliding. In other embodiment, the third filter 1 is stored within the second sheath 19 by longitudinal sliding.

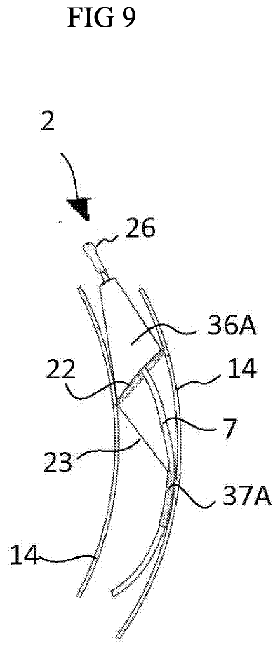

FIG. 9 is a side cross-sectional view of a second filter 2 positioned within the left carotid artery 14, according to an embodiment of the invention. The second filter 2 is a basket shaped filter. A first mesh 36A is impermeable to embolic materials, the carotid ring 22 help to keep the filter open and adjusted to the vessel wall. Radiological contrast from a second guidewire lumen 7A (not seen) is adapted to be injected through the tip 26. The support cable 23 is used to keep the second filter 2 stable and prevents it from rising up with blood pressure. A holder fixture 37A attaches the support cable 23 to the second conduit 7, according to an embodiment of the invention.

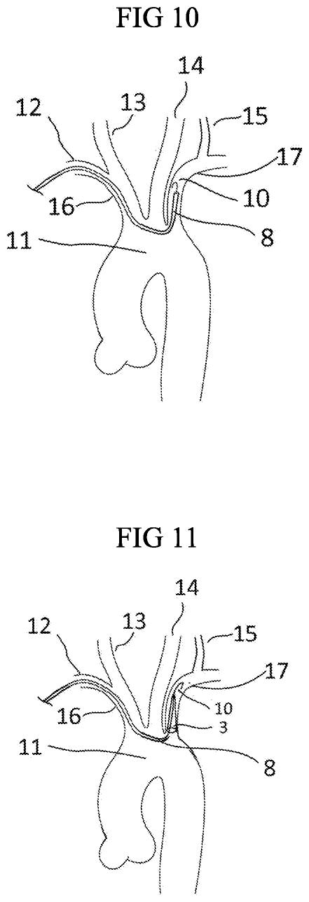

FIG. 10 is an illustration of the invention being used in the living body according to an embodiment of the invention. An aortic arch 11 is part of the human body; a right brachial artery 12, a right carotid artery 13, a left carotid artery 14, a left vertebral artery 15, a brachiocephalic trunk 16, a left subclavian artery 17 are also parts of the human body and are arteries whose blood stream irrigates the brain and are called here cerebral arteries.

The first guidewire 10 and the first conduit 8 are accessing the left subclavian artery 17 through the right brachial artery 12 and through the aortic arch 11 using the first guidewire 10 as a guide according to an embodiment of the invention.

FIG. 11 is an illustration of other embodiment of the invention showing the first filter 3 deployed in the left subclavian artery 17. The first guidewire 10 and the first conduit 8 are accessing the left subclavian artery 17 through the right brachial artery 12, according to an embodiment of the invention.

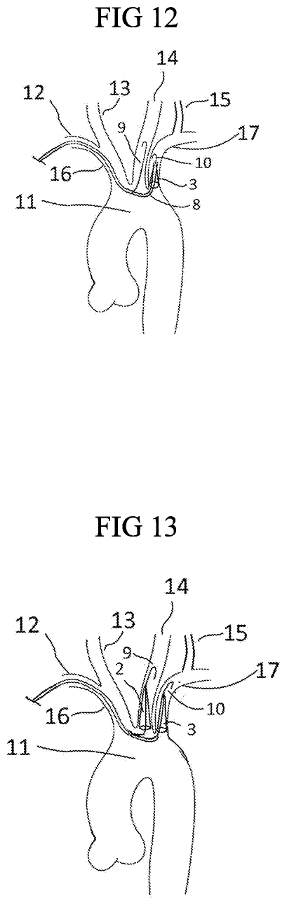

FIG. 12 is an illustration of other embodiment of the invention in which the first guidewire 10 is positioned in the left carotid artery 14 with the third filter 1 deployed in the left subclavian artery 17, according to an embodiment of the invention.

FIG. 13 is an illustration of other embodiment of the invention showing the second filter 2 deployed in the left carotid artery 14 and with the third filter 1 deployed in the left subclavian artery 17.

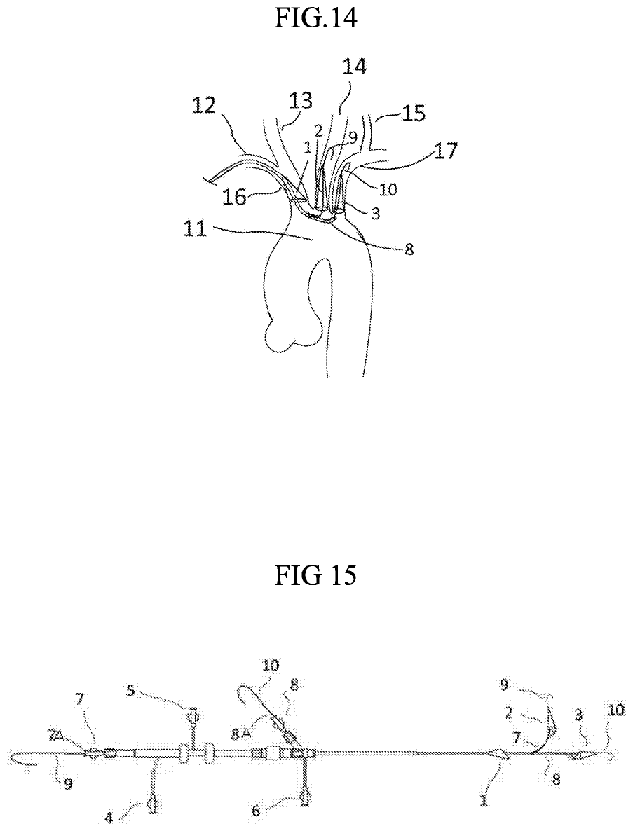

FIG. 14 is an illustration of other embodiment of the invention showing the first filter 3 deployed in the brachiocephalic trunk 16, the second filter 2 deployed in the left carotid artery 14 and with the third filter 1 deployed in the left subclavian artery 17. One aspect of the example is an endovascular three filters catheter 34 and a blood filtering method of use that captures and removes particles caused as a result of a surgical or endovascular procedures. The method includes a first filter 3 deployed in a first vessel within the patient's vascular system, a second filter 2 deployed in a second vessel within the patient's vascular system and a third filter 1 deployed in a third vessel within the patient's vascular system.

FIGS. 10-14, in sequence FIG. 10 to FIG. 14, also illustrates a method of implantation of the blood filtering catheter for cerebral embolic protection, according to an embodiment of the invention. This example illustrates a method for cerebral embolic protection using a three filters catheter 34 by the steps of: (a) deploying a filter in a first cerebral artery, (b) deploying a second filter in a second cerebral artery and (c) deploying third filter in a third cerebral artery, aiming to increase the level of particle protection.

In one embodiment the implantation of the three filters catheter 34 is performed by accessing the right brachial artery 12, using the first guidewire 10 as a guide. In other embodiment the three filters catheter 34 is progressed to the brachiocephalic trunk 16. In other embodiment the three filters catheter 34 is inserted directly into a right subclavian artery 40 (not seen).

In one embodiment the three filters catheter 34 is progressed to the aortic arch 11, the three filters catheter 34 has adequate curvature to easily access the left subclavian artery 17. In other embodiment the three filters catheter 34 insertion direction is controlled to easily access the left subclavian artery 17.

In this example the first guidewire 10 is progressing through the first guidewire lumen 8A and is progressing to the left sub clavicular artery 17, the first filter 3 is progressing and is deploying in the left subclavian artery 17. Alternatively, the first filter 3 is deploying in the left vertebral artery 15.

In this example the first filter 3 serves as an anchor to facilitate deploying of the second filter 2 in the left carotid artery 14. The second guidewire 9 is pushing and easily progressing to the left carotid artery 14, the three filters catheter 34 is progressing to the left carotid artery 14 and the second filter 2 is deploying in the left carotid artery 14. Generally, the third filter 1 is deploying in the brachiocephalic trunk 16. In this example all the cerebral arteries are protected from debris. Alternatively, the three filters catheter 34 is adapted to be positioning in the left vertebral artery 15. Alternatively, the first filter 3 is adapted to be collecting while maintaining the other filters deployed. Alternatively, the catheter is adapted to be implanting only with two filters, the third filter 1 and one of the two other filters in another cerebral artery. According to an embodiment of the invention.

The three filters catheter 34 is inserted and implanted at the site to capture embolisms and other foreign bodies and prevent its passage into the cerebral vasculature of the patient, to avoid or minimize thromboembolic disorders, such as a strokes.

FIGS. 14-10, in sequence FIG. 14 to FIG. 10, also illustrates the removing of the filters after termination of the endovascular or cardiac procedure, the first filter 3, the second filter 2 and the third filter 1 are collapsing and withdrawing from the arteries. This example also shows a method for cerebral embolic protection using a three filters catheter by the steps of: (a) removing a filter of a first cerebral artery, (b) removing a second filter of a second cerebral artery, (c) removing a third filter of a third cerebral artery, according to an embodiment of the invention. In an alternative embodiment, in the end of the surgical procedure, the second filter 2 containing debris is closed and brought to the third sheath 21, then the first filter 3 is closed and brought to the third sheath 21, then the third filter 1 is closed and brought to the second sheath 19, then the whole set is brought to the first sheath 18 and removed from the living body. In an alternative embodiment, one or two or three filters may be withdrawn from the filtration system and left inside the patient for a therapeutic period. According to an embodiment of the invention.

FIG. 15 is a side view of other embodiment of the three filters catheter for cerebral embolic protection. The second filter 2 is carried on the second conduit 7 having a second guidewire lumen 7A extending therethrough. A second conduit 7 having a second guidewire lumen 7A allows the user to precisely deliver the second filter 2 by advancing the second filter 2 over the second guidewire 9. In some embodiments the second conduit 7 is configured to have increased column strength to aid the advancement of the second filter 2. In some embodiments, the second conduit 7 includes a flexible portion to better position the second filter 2 within the vessel. In some embodiments, the first filter 3 is carried on a first conduit 8 having a first guidewire lumen 8A extending therethrough. A first conduit 8 having a first guidewire lumen 8A allows the user to precisely deliver the first filter 3 by advancing the first filter 3 over the first guidewire 10. The first conduit 8 is adapted to be configured to have increased column strength to aid advancement of the first filter 3. In some embodiments, the first conduit 8 includes a flexible portion to better position the first filter 3 within the vessel.

Additionally, the structures described herein are adapted to be embodied as integrated components or as separate components.

It will be apparent to one skilled in the art that the invention may be provided with some or all the mentioned features and components without departing from the spirit and scope of the invention. For purposes of comparing various embodiments, certain aspects and advantages of these embodiments are described. Not necessarily all such aspects or advantages are achieved by any particular embodiment. Thus, for example, various embodiments are adapted to be carried out in a manner that achieves or optimizes one advantage or group of advantages as described herein without necessarily achieving other aspects or advantages as can also be described or suggested herein. It will also be apparent to the skilled artisan that the embodiments described above are specific examples of a broader invention which may have greater scope than any of the singular descriptions taught. There may be many alterations made in the descriptions without departing from the spirit scope of the invention.

Although certain preferred embodiments and examples are disclosed, inventive subject matter extends beyond the specifically disclosed embodiments to other alternative embodiments and/or uses, and to modifications and equivalents thereof. Thus, the scope of the claims appended hereto is not limited by any of the particular embodiments described. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent.

1. A blood filtering catheter, comprising: three filters for cerebral embolic protection.
1.1. The catheter of claim 1, further comprising at least one guidewire lumen.
1.2. The catheter of claim 1, further comprising at least one basket shaped filter.
1.3. The catheter of claim 1, further comprising at least one filter made of a material impermeable to embolic materials.
1.4. The catheter of claim 1, further comprising means to control insertion direction of at least one filter.
1.5. The catheter of claim 1, further comprising at least one fluid lumen in an elongated catheter shaft, the fluid lumen having at least one fluid connection.
1.6. The catheter of in claim 1, further comprising at least one distal end of the catheter deflectable with respect to a longitudinal axis of the catheter in absence of any deflection.
1.7. The catheter of claim 1, further comprising at least one radiological mark.

2. A method for cerebral embolic protection using a three filters catheter, comprising:
(a) deploying a first filter in a first cerebral artery;
(b) deploying a second filter in a second cerebral artery; and
(c) deploying a third filter in a third cerebral artery.

3. A method for cerebral embolic protection using a three filters catheter, comprising:
(a) removing a first filter of a first cerebral artery;
(b) removing a second filter of a second cerebral artery; and
(c) removing a third filter of a third cerebral artery.
